(19) United States
(12) Patent Application Publication
(10) Pub. No.: US 2018/0050005 A1
(43) Pub. Date: Feb. 22, 2018

(54) Concentrated Solution of 17-Hydroxydocosahexaenoic Acid

(71) Applicant: Janssen Pharmaceutica NV, Beerse (BE)

(72) Inventors: Thomas M DiMauro, Southboro, MA (US); Kevin Wildenhaus, Plymouth, MI (US)

(73) Assignee: Janssen Pharmaceutica NV, Beerse (BE)

(21) Appl. No.: 15/354,114

(22) Filed: Nov. 17, 2016

Related U.S. Application Data
(60) Provisional application No. 62/375,676, filed on Aug. 16, 2016.
(60) Provisional application No. 62/402,439, filed on Sep. 30, 2016.

(51) Int. Cl. A61K 31/202; A61K 9/16; A61K 9/08; C11C 1/04; C11C 1/08; C11B 7/00; C12P 7/64; B01D 15/08

(52) U.S. Cl. CPC A61K 31/202; A61K 9/167; A61K 9/08; C11C 1/045; B01D 15/08; C11B 7/0075; C12P 7/6409; C12Y 301/01004; C11C 1/08

(57) ABSTRACT

A method of treating a mother having postpartum depression, comprising administering to the mother a composition comprising a plurality of mixed self-assemblies comprising:
i) at least 50 wt % of a soyasaponin, and
ii) allopregnanolone.

### CONTINUING DATA

This application claims priority from co-pending provisional applications U.S. Ser. No. 62/375,676, filed Aug. 16, 2016 (DiMauro et al.), entitled “Self-Assemblies Comprising Intercalated Natural GABA(A) Delta Agonists” (Docket No. PRD3425USPSP) and U.S. Ser. No. 62/402,439, filed Sep. 30, 2016 (DiMauro et al.), entitled “Tailored Novel Nutriceutical Solutions to the Heterogeneous Phenotypes of Perinatal Depression” (Docket No. PRD3425USPSP1), the specifications of which are incorporated by reference in their entireties.

### BACKGROUND OF THE INVENTION

The loving connection between a mother and her baby is a special bonding that can benefit the baby not only in the present, but also well into the future. Bonding brings the mother and child closer together, and this positive attachment can enhance the baby's wellbeing and later development. Because a healthy bond between the mother and her newborn infant is crucial to the proper development of the child, loving efforts to strengthen that bond are highly valued. Some of the ways in which a healthy mother can show love for her child and promote this bonding is by experiencing joy at her child's smile and by providing appropriate attention to her child's needs.

It has been estimated that over 700,000 mothers are afflicted with postpartum depression (PPD) each year in the United States. PPD is considered to be a major depression, and is characterized by standard depressive features. Typical PPD symptoms include non-responsiveness towards the infant's needs and an absence of joy that is normally associated with healthy parent-child interaction and attachment. Because the first months of life are a critical period for an infant's proper cognitive and emotional development, the lack of attachment and attention towards the infant shown by the PPD mother may cause undesired effects in the child's future behaviors.

During pregnancy, the hormonal balance in the healthy expectant mother is such that she experiences extremely high levels of estrogen throughout her body. These levels of estrogen in the expectant mother may be up to 100 times the normal level. After the birth of the child, the estrogen level in the new mother rapidly decreases over the course of a few days and returns to the normal level of estrogen. Estrogen has been found to be critical to many normal neuronal processes, and has been positively associated with serotonin levels in the brain and brain plasticity. Therefore, and without wishing to be tied to a theory, it is believed that PPD may be caused by an extra-sensitive response in a subset of new mothers to the rapid withdrawal of estrogen from the mother's system.

Antidepressants are often one of the first lines of therapy against PPD. Conventional antidepressants such as tricyclics and selective serotonin reuptake inhibitors (SSRIs) are commonly prescribed for PPD. However, there are many problems associated with the use of these conventional antidepressants for PPD. First, these conventional antidepressants typically alleviate the PPD condition in no more than about 80% of the patients taking them. Second, even when successful, these conventional antidepressants typically take up to 8 weeks be effective. Third, the PPD mother can expect to experience the typical side effects associated with tricyclics and SSRIs. Side effects associated with SSRI use include insomnia, weight gain and sexual dysfunction.

In addition, it has been found that virtually all of these conventional antidepressants are found in the mother's milk, and may be transferred to the infant during nursing. There has been little data on the effect of the nursing mother's antidepressant use upon the child's mental development. Rather than demonstrating safety, the literature appears to conclude that the risk to the nursing child posed by the mother's antidepressant use is outweighed by the risks associated with untreated PPD. However, in some cases, the transfer of some particular antidepressants to mother's milk has been so significant that some investigators have concluded that those particular antidepressants should be avoided by nursing mothers.

Sertraline (Zoloft) and paroxetine (Paxil) are the first-line antidepressants for treating PPD (Berle, Curr. Womens Health Rev. 2011 February; 7(1):28-34). No long term studies on the effects of these antidepressants on infants who receive their mother's milk.

It has recently been reported by Sage Pharmaceuticals that their compound, SAGE-547, showed efficacy in a small double blind human trial. However, the SAGE-547 must be administered by an intravenous method, and so poses a problem with day-to-day compliance.

Therefore, one goal is to provide a GABA(A) delta agonist that can be administered non-invasively.

### SUMMARY OF THE INVENTION

It has now been appreciated that there exists in nature several GABA(A) agonists that possess a self-assembling quality. This quality allows the skilled artisan to make self-assembled structures including gels, micelles, multi-lamellar vesicles (MLVs) and liposomes from the natural GABA(A) delta agonists.

Because LMVs, micelles and liposomes can be administered non-invasively through the oral, intranasal or pulmonary routes, the present compositions present an advantage over the prior art intravenous compositions.

In some embodiments, the natural GABA(A) delta agonist is presented in the form of micelles. Micelles provide an advantage in that they can be orally administered and that their small size evades detection by macrophages and so provides for an extended circulation time in the human vasculature.

In some embodiments, the natural GABA(A) delta agonist is presented in the form of liposomes. It is believed that the liposomal form provides an advantage during pulmonary administration of the GABA(A) delta composition. Liposomes are generally on the order of 100-200 nanometers (and so are categorized as fine particles), while micelles are much smaller at about 10-20 nm (and so are categorized as ultrafine particles). Because a substantially larger fraction of micelles are exhaled after pulmonary administration, liposomes provide an advantage (over micelles) in that their relatively larger size provides a much more efficient pulmonary administration. Liposomes can also deliver hydrophilic molecules housed in their aqueous cores.

In some embodiments, the natural GABA(A) delta agonist is presented in the form of multi-lamellar vesicles (MLVs). It is believed that the MLV form provides an advantage during pulmonary administration of the GABA(A) delta composition. MLVs can be made to a size on the order of a few microns. Because a substantially larger fraction of micelles and liposomes are exhaled after pulmonary administration, MLVs provide an advantage (over micelles and liposomes) in that their relatively larger size provides a much more efficient pulmonary administration.

Therefore, in some embodiments, there is provided a composition comprising a plurality of mixed self-assemblies comprising:

i) at least 50 wt % of a cyclic molecule, and

ii) at least 5 wt % of a natural GABA(A) delta agonist intercalated therein.

In other embodiments, there is provided a composition comprising a plurality of mixed self-assemblies comprising:

i) at least 50 wt % of an uncharged molecule, and

ii) at least 5 wt % of a natural GABA(A) delta agonist intercalated therein.

In other embodiments, there is provided a composition comprising a plurality of mixed self-assemblies comprising:

iii) at least 50 wt % of an glycosylated molecule, and

iv) at least 5 wt % of a natural GABA(A) delta agonist intercalated therein.

Preferably, each has at least a trihexacyclic structure.

In other embodiments, there is provided a composition comprising a plurality of mixed self-assemblies comprising:

v) at least 50 wt % of an phenolic molecule, and

vi) at least 5 wt % of a natural GABA(A) delta agonist intercalated therein.

Preferably, each has a biphenyl structure, more preferably a biphenolic structure.

### DETAILED DESCRIPTION OF THE INVENTION

For the purposes of the present invention, a GABA(A) delta agonist increases a GABA(A) current at least 10% at 100 uMol/L.

In some embodiments, the self-assembled structure is selected from the group consisting of a micelle, a liposome, an MLV, and a solid lipid nanoparticle.

In some embodiments, the GAAB delta agonist is derived from a plant. In others, the agonist is endogenous to mammals. In others, the agonist is endogenous to humans.

In some embodiments, each of the cyclic structure and the agonist contains a biphenyl structure. These common aromatic structures allow the agonist to nest within opposed biphenyls of the cyclic superstructure (i.e., the biphenyl of the agonist intercalates between the biphenyls of the cyclic superstructure). This intercalation is carried out due to the pi-pi bonding between the aromatic components of the biphenyl structures.

In some embodiments, the cyclic molecule is ellagic acid, a urolithin, a punicallagin or mixtures thereof.

In some embodiments, the cyclic molecule is ellagic acid, a urolithin or mixtures thereof and the agonist is a lignan selected from the group consisting of honokiol or magnolol, or mixtures thereof. Preferably, the cyclic molecule is ellagic acid and the agonist is a lignan selected from the group consisting of honokiol or magnolol, or mixtures thereof.

In some embodiments, the cyclic molecule is unsaturated. In some embodiments, each of the unsaturated cyclic molecule and the agonist has at least three cyclohexylic rings. These common rings allow the agonist to nest within opposed rings of the unsaturated cyclohexylic superstructure.

In some embodiments, the cyclic molecule is a saponin and the agonist is a neurosteroid. Preferably, the saponin selected from the group consisting of a soyasaponin, quillaja saponin and a ginsenoside, and the neurosteroid is selected from the group consisting of allopregnanolone, THDOC and progesterone.

In some embodiments, the self-assembly contains at least 5 mol % neurosteroid, preferably at least 10 mol %, more preferably at least 20 mol %, more preferably at least 30 mol %, more preferably at least 40 mol %.

In some embodiments, the self-assemblies comprise at least 15 wt % of the natural GABA(A) delta agonist. Preferably, the self-assemblies comprise at least 30 wt % of the natural GABA(A) delta agonist.

In some embodiments, the self-assemblies comprise at least 65 wt % of the cyclic molecule, preferably, at least 80 wt % of the cyclic molecule.

In some embodiments, the cyclic molecule is at least bicyclic (i.e., has at least two rings).

Allopregnanolone and THDOC are highly potent GABA(A) delta agonists. THDOC increases the GABA current by at least 700% at a concentration of 1 uMol/L. See Wohlfarth, J. Neurosci., 2002, 22, 5, 1541-9. Allopregnanolone potentiates rat cerebellar GABA delta subunits in the nanomolar range. Fodor, Neurosci. Lett., 2005, 383, (1-2), 127-130.

Allopregnanolone is endogenous to human and rises in plasma concentration during pregnancy from less than 5 ng/ml to about 50 ng/ml. Luisi, J. Clin. Endocrinol. Metab., 2000, July 85, 7, 2429-33. Therefore, it can be administered safely to a mother without concern for the health of the breastfeeding infant.

THDOC is endogenous to human and exists in concentrations as high as 0.5 ng/ml in the plasma of humans. Brambilla, Psychiatry Research, 135, 2005, 185-190. Therefore, it can be administered safely to a mother without concern for the health of the breastfeeding infant.

Progesterone is endogenous to humans and rises in plasma concentration during pregnancy from less than 10 ng/ml to about 150 ng/ml. Luisi, J. Clin. Endocrinol. Metab., 2000, July 85, 7, 2429-33. Therefore, it can be administered safely to a mother without concern for the health of the breastfeeding infant.

In one embodiment, a self-assembled allopregnanolone/soyasaponin mixed micellar structure is made substantially in accordance with the recipe for making an ginsenoside micelles disclosed in Xiong, Intern. J. Pharmaceutics, 360 (2008) 191-196. In one such prophetic embodiment, a series of working solutions are prepared by dissolving a 10 mol % allopregnanolone/90 mol % soyasaponin mixture in water and physiologic saline to produce 0.1-0.6 mg/ml solutions. These working solutions are then filtered through a 0.8 um filter. Surface tension is then measured to identify the critical micellar concentration CMC. The micellar solutions are then subject to evaporation to obtain dry mixed micelles.

In some embodiments, the allopregnanalone is disposed in an alcohol solution (such as 95 w/o ethanol) prior to its mixing with the saponin, as doing so increases the solubility of the allopregnanalone in the solution and allows for its facile intercalation during the fabrication of the saponin self-assembly.

In one embodiment, a self-assembled allopregnanolone/ginsenoside mixed micellar structure is made substantially in accordance with the recipe for making an ginsenoside micelles disclosed in Xiong, Intern. J. Pharmaceutics, 360 (2008) 191-196. In one such prophetic embodiment, a series of working solutions are prepared by dissolving a 10 mol % allopregnanolone/90 mol % ginsenoside mixture in water and physiologic saline to produce 1-100 mg/ml solutions. These working solutions are then filtered through a 0.8 um filter. Surface tension is then measured to identify the critical micellar concentration CMC. The micellar solutions are then subject to evaporation to obtain dry mixed micelles.

In one embodiment, a self-assembled allopregnanolone/quillaja saponin mixed micellar structure is made substantially in accordance with the recipe for making an ginsenoside micelles disclosed in Xiong, Intern. J. Pharmaceutics, 360 (2008) 191-196. In one such prophetic embodiment, a series of working solutions are prepared by dissolving a 10 mol % allopregnanolone/90 mol % quillaja saponin mixture in water and physiologic saline to produce 1-100 mg/ml solutions. These working solutions are then filtered through a 0.8 um filter. Surface tension is then measured to identify the critical micellar concentration CMC. The micellar solutions are then subject to evaporation to obtain dry mixed micelles.

In the neurosteroid/saponin embodiments, a thin film hydration method can be used to make multi-lamellar vesicles (MLVs) and liposomes.

In order to make self-assembled MLVs, DBC/TTC is first dispersed in an organic solvent in a rotatory evaporator flask, and the solvent is evaporated to leave a thin film on the bottom of the flask. The film is then hydrated with water, and the flask is gently agitated to form the MLVs.

In another embodiment, a self-assembled MLV is made as above, and then shear is imparted upon the solution. In some embodiments, the shear is produced by a high speed blender. In other embodiments, the shear is produced by a commercially available ultrasonic cleaner.

The self-assembled allopregnanolone/ginsenoside mixed self-assembled structures are useful because ginsenosides are also known as useful for treating diabetes. Thus, with a single structure the clinician can treat both postpartum depression and gestational diabetes in the same mother.

It has been reported that self-assembled ginsenoside micelles can be tuned to have release rates from days to months. Xiong, Int. J. Pharm. Aug. 6, 2008; 360(1-2):191-6. The tuning is performed by varying the concentration of the ginsenoside in the initial solution, with higher concentrations leading to slower release rates. Without wishing to be tied to a theory, it is believed self-assembled vesicles consisting essentially of a glycosylated saponins (such as soyasaponin) can likewise be tuned to have release rates from days to months, with the tuning being performed by varying the concentration of the local anaesthetic in the initial solution, with higher concentrations leading to slower release rates.

Therefore, in some embodiments that provide for extended release rates, the self-assembled micelle of allopregnanolone/soyasaponin is made by dispersing allopregnanolone/soyasaponin in water at a concentration above 1 mg/ml, preferably above 5 mg/ml, preferably above 20 mg/ml, preferably above 30 mg/ml, preferably above 50 mg/ml, preferably above 70 mg/ml, preferably above 80 mg/ml, preferably above 90 mg/ml, preferably above 100 mg/ml.

Likewise, in some embodiments that provide for extended release rates, the self-assembled micelle of allopregnanolone/ginsenoside is made by dispersing allopregnanolone/ginsenoside in water at a concentration above 1 mg/ml, preferably above 5 mg/ml, preferably above 20 mg/ml, preferably above 30 mg/ml, preferably above 50 mg/ml, preferably above 70 mg/ml, preferably above 80 mg/ml, preferably above 90 mg/ml, preferably above 100 mg/ml.

Xiong teaches that loading determines release rate. In some embodiments, the loading of the self-assembly is targeted to provide a release rate that corresponds to about a 100% release in about 24 hours and a 50% rate at about 12 hours. This loading and corresponding rate would enable the mother to take only one dose a day (and thereby promote compliance more than a multiple-dose-per-day routine) while still enabling a habit-forming routine of taking one dose per day (thereby promote compliance more than a one-dose-every-few-days routine).

According to Alexeev, Neuropharmacology. 2012 June; 62(8):2507-14, honokiol and magnolol are very potent GABA(A) delta agonists, as each increases the GABA current by at least about 800% at a concentration of about 10 uMol/L.

Honokiol and magnolol are found in the fruit and bark of the magnolia tree. Honokiol has been suggested to treating post-natal pain in infants. Woodbury, J Nat Prod. Nov. 25, 2015;78(11):2531-6. Therefore, it appears to be a good candidate for safe administration to a mother without much concern for the health of the breastfeeding infant.

In one embodiment, honokiol/ellagic acid self-assemblies are made substantially in accordance with the recipe for making an ellagic acid self-assemblies disclosed in Frayne, Materials Express, 2, 4, 2012 335-343. In one such prophetic embodiment, 10% honokiol/90% ellagic acid assemblies are prepared in aqueous solution at pH 7. A stock solution of 50 mL of 9 mM ellagic acid and 1 mM honokiol was dissolved in 0.1 M NaOH and filtered. To the filtrate, 0.1 M solution of citric acid was added to adjust the pH value of the solution to 8. In some embodiments for making elongated sandwich structures, the above mixture is allowed to grow for a maximum of 15 minutes (thereby preventing polymerization from occurring). The formed assemblies are sonicated for thirty minutes, washed and deionized with water, and centrifuged twice at 15000 rpm before further analysis.

The self-assembled honokiol/ellagic acid mixed micellar structures are useful because ellagic acid is also known as useful for treating diabetes. Thus, with a single structure the clinician can treat both postpartum depression and gestational diabetes in the same mother.

These self-assembled combinations can be administered through oral, intranasal, buccal or pulmonary routes. The pulmonary route is preferred, as above.

It is further recognized that there are many other additional combinations of natural molecules (both phytochemicals and metabolites) whose first molecule has sufficient surfactant-like quality to form the superstructure of self-assemblies such as micelles, MLVs, LUVs, liposomes, cylinders, fibers and discs, and a second active molecule that has sufficient structure to nest neatly in the superstructure to provide enhanced bonding and therefore a slow release rate. A listing of some of these combinations of molecules is provided in Table I. These self-assembled combinations can be administered through oral, intranasal, buccal or pulmonary routes. The pulmonary route is preferred, as above.

Each of myrtenol and verbenol is also a GABA delta agonist at 100 uM. Van Brederode, Neuroscience Letters, 628, (2016) 91-97.

Each of myrtenol and verbenol is a major metabolite of alpha-pinene (pine nuts). Schmidt, Arch. Toxicol., Dec. 17, 2015 and has been determined to be Generally Regarded as Safe (GRAS) by the FDA (Duke, 2000)

In one embodiment, a self-assembled verbenol or myrtenol micelle is made substantially in accordance with the recipe for making a camphor micelle disclosed Turina, Biophysical Chemistry, 122, 2006, 101-113. In one such prophetic embodiment, verbenol is dispersed in water at a concentration above 0.01 mM.

In another embodiment, a self-assembled verbenol liposome is made by first providing verbenol is dispersed in an organic solvent, rotary evaporating the solvent to form a thin film on the flask bottom, hydrating the film with agitation to form MLVs. Liposomes can be made by further imparting shear upon the solution. In some embodiments, the shear is produced by a high speed blender. In other embodiments, the shear is produced by a commercially available ultrasonic cleaner. LUVs can be made as above.

It is further recognized that there are many other additional natural molecules (both phytochemicals and metabolites) that (like the verbenol and myrtenol molecules discussed above) also have sufficient surfactant-like quality to form self-assemblies such as micelles, MLVs, LUVs, liposomes, cylinders, fibers and discs) all by themselves. That is, the self-assembly consists essentially of the natural molecule. A listing of some of these molecules is provided in Table II. These self-assemblies can be administered through oral, intranasal, buccal or pulmonary routes. The pulmonary route is preferred, as above. In some embodiments, the self-assembly consists essentially of a phytochemical. In some embodiments, the self-assembly consists essentially of an endogenous molecule. In some embodiments, the self-assembly consists essentially of a human metabolite.

The thin film hydration method can be used to make MLVs, large unilamellar vesicles (LUVs) and liposomes from the combinations and molecules listed in Tables I and II.

In order to make self-assembled MLVs, the surfactant is first dispersed in an organic solvent in a rotatory evaporator flask, and the solvent is evaporated to leave a thin film on the bottom of the flask. The film is then hydrated with water, and the flask is gently agitated to form the MLVs.

In another embodiment for making LUVs, a self-assembled MLV is made as above, and the MLVs are then extruded through a properly sized filter to form the LUV.

In another embodiment, a self-assembled MLV is made as above, and then shear is imparted upon the solution to produce liposomes. In some embodiments, the shear is produced by a high speed blender. In other embodiments, the shear is produced by a commercially available ultrasonic cleaner.

In some embodiments, oxytocin or an analog thereof is provided in the water core of the liposome in an amount effective for treating PPD.

TABLE I

| | | | Rationale for self- |
|---|---|---|---|
| Molecule | Mode of Action | Application | assembling behaviour |
| Magnolol | GABA(A) delta agonist | Postpartum depression; | Intercalation in ellagic |
| | | cancer (HIF) | acid, which forms a |
| | | | sandwich. Barnaby, J. |
| | | | Nanosci. Nanotech., |
| | | | 2011, Sept., |
| | | | 11,9,7579-86 |
| Honokiol | GABA(A) delta agonist | Postpartum depression;; | Intercalation in ellagic |
| | | cancer | acid, which forms a |
| | | | sandwich. Barnaby, J. |
| | | | Nanosci. Nanotech., |
| | | | 2011, Sept., |
| | | | 11,9,7579-86 |

TABLE II

| | | | Rationale for self- |
|---|---|---|---|
| Molecule | Mode of Action | Application | assembling behaviour |
| Borneol | GABA(A) agonist | depression | Looks like camphor |
| Sulforaphane | Nrf2 | Autism; breast cancer | Similarity to octyl methyl |
| | | metastasis; COPD | sulfoxide. (loyota, J |
| | | | Colloid Interface Sci. 2006 |
| | | | Jul 1; 299(1): 428-34) |
| Perillyl alcohol | Ras inhibitor | Glioblastoma | Similarity to menthol |
| | | multiforme | |
| Allicin | Antibiotic | Ear infection | Similarity to sodium |
| | | | ricinolate (Shinde, J. |
| | | | Phys. Chem., 1992, 96, |
| | | | 5160-5) |
| 2-arachidonoyl glycerol | Endogenous cannabinoid | Postpartum depression; | Similarity to ceramide, |
| (2-AG) | | | which forms a liposome. |
| | | | Park, Biochem Biophys |
| | | | Res Commun. 2013 |
| | | | Jun 7; 435(3): 361-6 |
| Anandamide | Endogenous cannabinoid | Postpartum depression; | Similarity to ceramide |
| Oleamide | Endogenous cannabinoid | Postpartum sleep | Similarity to ceramide |
| 17-hydroxy | Precursor to | Postpartum depression; | Similarity to DHA. |
| docosahexaenoic acid | neuroprotectin | diabetes | Mooibroek, Int J Radiat |
| | | | Biol Relat Stud Phys |
| | | | Chem Med. 1982 |
| | | | Dec; 42(6): 601-9. |
| | | | Similarity to ricinoleic |
| | | | acid. |
| Hesperidin | Upregulates BDNF | Postpartum | Glycosylated flavonoid |
| | | depression; diabetes | |
| 1,8 cineole | Brain wave | Mood elevation | Turina, Biophys. Chem., |
| | | | 2006 July 20; 122(2), 101-13 |
| Ganglioside GM3 | Self-assembling liposome | Postpartum depression | Similarity to lecithin |
| | housing oxytocin | | |
| Hyperoside | Antidepressant; Beta2- | Postpartum depression; | Glycosylated flavonoid |
| | adrenergic blocker (Beta- | metastatic breast cancer | |
| | blocker) | | |
| Quercetin-3-glucuronide | Beta2-adrenergic | Metastatic breast | Similarity to hyperoside |
| (Q3G) | blocker (Beta-blocker) | cancer | |
| Rutin | Precursor of hyperoside | Metastatic breast | Double glycosylated |
| | | cancer | flavonoid |
| Propranolol | Beta2-adrenergic | Metastatic breast | Classic polar cationic |
| | blocker (Beta-blocker) | cancer; hypertension | head and lipophilic tail |
| Crocetin/crocin | antidepressant | Postpartum depression | Bolaamphiphile with |
| | | | similar diacid structure in |
| | | | Zhang, J. Colloid interface |
| | | | Sci., 2003 May |
| | | | 15,261,2,417-22. |
| 10-hydroxydecenoic acid | Promotes neurogenesis | Antidepressant; cancer | Hydroxyl & carboxyl ends |
| | of neural stem cells | | |
| Bisabolol oxide A | | | Intercalation with |
| | | | soyasaponin/gensenoside |
| Sphinosine-1-phosphate | Chemotactic agent for | Intradiscal injection for | Similarity to lecithin |
| (S1P) | stem cells | DDD; fusion agent | |
| 2-hydroxyoleic acid | Anticancer agent | Glioma, leukemia, | Classic polar head- |
| | | breast cancer and colon | hydrophobic tail |
| | | cancer | surfactant structure |
| Oleuropein | Anticancer | Breast cancer | Classic glycosidic head- |
| | | | hydrophobic tail |
| | | | surfactant structure |

Without wishing to be tied to a theory, it is believed that PND is not a single condition, but rather is a heterogeneous disorder consisting of at least six different phenotypes. Presented herein is a portfolio of novel products, each of which provides a tailored pharmaceutical treatment for at least one of the six PND phenotypes. Because there is sensitivity to the possibility of transferring these products to the infant through breastfeeding, the tailored solutions use only molecules having extremely high safety profiles (i.e., nutriceuticals, their metabolites or endogenous molecules).

The Table III below provides a description of at least some of the hypothesized phenotypes along with tailored solutions for the phenotypes.

TABLE III

| Phenotype | Description | Tailored solution |
|---|---|---|
| 1a | Postpartum: GABA delta receptor expression | Allopregnanolone/soyasaponin |
| | does not sufficiently rebound after delivery | mixed self-assembly |
| 1b | Antenatal: GABA delta receptor expression | Honokiol/Ellagic acid mixed |
| | is too stunted during pregnancy | self-assembly |
| 2 | Postpartum: Failure to modulate estrogen | Equol/soyasaponin mixed self- |
| | levels after delivery | assembly: |
| 3 | Postpartum: Without wishing to be tied to a | Zinc chelated Oxytocin |
| | theory, it is believed that oxytocin levels do | Micelle |
| | not increase postpartum with a | |
| | corresponding decrease in cortisol as desired. | |
| | It is hypothesized that the elevated cortisol | |
| | level blocks the benefits of oxytocin, so one | |
| | solution is to lower stress levels to allow | |
| | oxytocin to work. | |
| 4a | Antenatal: Underlying inflammation leads to | Concentrated 17-OH DHA |
| | pain, poor sleep, anxiety and rumination. | |
| | +++++++ | +++++++ |
| | Gestational diabetes mellitus is often present | 17-OH DHA is also effective |
| | along with this antenatal depression | to prevent or manage |
| | phenotype | gestational diabetes mellitus |
| 4b | Postpartum: inflammation stemming from a | Treat with a chelated Trkb- |
| | complicated delivery does not resolve, | agonizing hydroxyflavone |
| | thereby leading to pain, poor sleep, anxiety | and/or a Trkb-agonizing |
| | and rumination, but more acutely and | mixed self-assembly |
| | intensely than phenotype 4a. | |

Allopregnanolone/soyasaponin mixed self-assembly: Earlier this year, Sage Pharmaceuticals announced very positive results for their phase II trial of intravenous allopregnanolone (SG-547) for postpartum depressed (PPD) mothers. Although encouraging, the requirement of an intravenous administration makes the Sage treatment inconvenient at best and likely subject to frequent noncompliance.

We have developed a treatment involving a mixed micelle of allopregnanolone and soyasaponin. Soyasaponin, which is present in soy infant formula (Fonseca, Food Chem. Jan. 15, 2014; 143:492-8) and so has a demonstrated safety profile, is also a surfactant capable of forming the superstructure of a micelle (DeCroos, Food Chemsitry 101, 2007, 324-323). We have made the novel observation that allopregnanolone and soyasaponin share common structure (see below). Because of this commonality of structure, it is believed that allopregnanolone will intercalate into the soyasaponin superstructure. This intercalation will produce enhanced bonding between the allopregnanolone and the soyasaponin superstructure, and thereby cause allopregnanolone to release from the micelle at a very slow rate that will allow for once-a-day administration.

Honokiol/Ellagic acid mixed self-assembly: One hypothesis of postpartum depression (PPD) is that the mother's GABA delta receptors in her brain fail to rebound after birth (Maguire, Pyschoneuroendocrinology, 2009, December 34(Suppl) S84-S90), and that the subsequent PPD can be ameliorated with the administration of a GABA delta agonist (Maguire, Neuron, 2008 July 31, 59(2) 207-213). We have developed a treatment for this phenotype involving an ellagic acid self-assembly intercalated with honokiol. Honokiol, which is present in magnolia bark and has been proposed as a treatment for infant pain (Woodbury, J Nat Prod. Nov. 25, 2015; 78(11):2531-6), is a highly potent GABA delta agonist that increases in vitro GABA currents about 900% at 10 μM (Alexeev, Neuropharmacology, 2012 June, 62(8), 2507-2514). Ellagic acid, which is present in strawberries and has been declared to be GRAS by the FDA, is a biphenyl structure that can form strong pi-pi bonds with other ellagic acid molecules (Frayne, Mater. Express, 2, 4, 2012, 335-343) and so can self-assemble (Barnaby, J. Nanosci. Nanotechnol. 2011 September, 11(9), 7579-86). We have made the novel observation that honokiol and ellagic share a common biphenyl structure having a plurality of hydroxyls extending therefrom. Because of this commonality, it is believed that honokiol will intercalate into the ellagic acid superstructure and thereby release from the ellagic acid self-assembly at a very slow rate.

Equol/soyasaponin mixed self-assembly: It has been reported that DNA methylation associated with PPD risk correlated significantly with estrogen-induced DNA methylation change, suggesting an enhanced sensitivity to estrogen-based DNA methylation reprogramming exists in those at risk for PPD (Guintivano, Mol. Psychiatry, 2014 May; 19(5):560-7), and further suggesting that estrogen can be therapeutic for some mothers with this PPD phenotype. However, concern for possible cancer-related side effects of estrogen has stunted its use. The isoflavanol Equol is a soy metabolite that is selective for the beta estrogen (non-cancer) receptor (ERβ) (Sareddy, Chin. J. Nat. Med., 2015 November; 13(11):801-7), and so does not carry a cancer risk. Equol is produced by the ingestion of soy formula, and is thought to have an excellent safety profile. We have made the novel observation that equol and soyasaponin share common structure, and so surmise that equol will intercalate into the soyasaponin superstructure and thereby release from the micelle at a very slow rate.

Zinc-chelated Oxytocin: Although some studies report the benefits of oxytocin for PPD mothers, its failure to cross the blood brain barrier (Chapman, Pharm Res. 2013 October; 30(10):2475-84) (thereby requiring an intranasal route of administration), and its short (˜6.8 minute) half-life (Paccamonti, Equine Vet J. 1999 July; 31(4):285-8), thereby requiring multiple dosings per day, prevent its more extensive use. Although oxytocin is not considered to be amphiphilic, we have made the novel observation that several journal articles show diagrams of zinc-chelated oxytocin appearing to have a surfactant-like distribution of hydrophilic and hydrophobic sites. These articles include:
a) Wyttenbach, J. Am. Chem. Soc., 2008, 130, 5993-6000. Note in FIG. 9c of Wyttenbach the clustering of the Ile, Tyr, Leu and Pro hydrophobic residues in the upper part of the chelate and the clustering of the Asn and Gln hydrophilic residues in the lower part of the chelate;
b) Fuller, J. Am. Soc. Mass Spectrom., 2016, 27, 1376-82. Note in FIG. 4 of Fuller the clustering of the Pro, Leu, Tyr and Ile hydrophobic residues around the lower right part of the figure, and the clustering of the hydrophilic Glu and Asn residues in the upper left part of the figure; and
c) Liu, J. Am. Chem. Soc., 2005, 127, 7, 2024-5. Note in FIG. 2c of Liu the clustering of the Ile, Tyr and Leu hydrophobic residues around the upper right part of the figure and the clustering of the Glu, Asn and Gly-NH₂ residues around the lower left part of the figure.

Moreover, it has been reported that this zinc-chelated oxytocin binds better to the OXT receptor better than oxytocin itself (Liu, J. Am. Chem. Soc., Feb. 23, 2015; 127(7):2024-5). We have developed a phosphatidylcholine-based sustained release device that exploits the surfactant-like nature of the zinc-chelated oxytocin, in which the zinc-chelated oxytocin intercalates into a standard phosphatidylcholine micelle. Because the zinc-chelated oxytocin has hydrophilic and hydrophobic regions that will respectively bond to the hydrophilic and hydrophobic parts of the phosphatidylcholine micelle, it will have greatly enhanced bonding to the micelle superstructure and thereby provide a slower release rate therefrom.

In other embodiments, the zinc-chelated oxytocin forms a self-assembly selected from the group consisting of a micelle, a liposome or a multi-lamellar vesicle.

Concentrated 17-OH DHA: 17-hydroxy docosahexaenoic acid (17-OH DHA) is a highly lipophilic fish oil metabolite and the metabolic precursor to neuroprotectin (Basselin, J Lipid Res. 2010 May; 51(5):1049-56), which is a potent anti-inflammatory that strongly upregulates bcl-2 in neurons (Bazan, J. Lipid Research, 51, 2010, 2018-2031 and Mukherjee, PNAS USA. Jun. 1, 2004; 101(22)). Because bcl-2 upregulation is thought to enhance synaptic plasticity (Manji, Biol Psychiatry. Apr. 15, 2003; 53(8):707-42.) and inflammation is one biomarker of one antenatal phenotype of PND (Roomruangwong, Mol. Neurobiol., Feb. 5, 2016), administering 17-OH DHA to an antenatally depressed mother should be beneficial towards alleviating that expectant mother's antenatal depression. Moreover, rat studies have shown 17-OH DHA to alleviate the symptoms of diabetes (Neuhofer, Diabetes. 2013 June; 62(6):1945-56). Lastly, 17-OH DHA has been found in mother's milk (Weiss, Lipids Health Disease, 2013, 12, 89) thereby verifying its safety. Therefore, 17-OH DHA looks to be an excellent candidate for administration to an antenatally depressed mother, especially one who suffers from gestational diabetes.

A. Zinc 17-OH DHA Chelate

We have developed a first novel process for concentrating 17-OH DHA from cow's milk. First, we start with the widely-available milk fat fraction that is a byproduct of the production of skim milk. Next, we have made the novel observation that 17-OH DHA has great structural similarity to ricinoleic acid, and believe that the chelated complex zinc ricinoleate has enhanced water solubility. Accordingly, we believe 17-OH DHA will form a chelate with zinc substantially in the same way that ricinoleic acid forms a chelate with zinc, and that the chelated 17-OH DHA complex will likewise have anenhanced water solubility. We thus propose to concentrate 17-OH DHA from the fat fraction by its zinc chelation and subsequent movement of the chelate into an aqueous phase. The novel components of the resulting aqueous phase (which likely also contains anti-inflammatory, chelatable resolvins and lipoxins) can be marketed as a 17-OH DHA-rich product.

B. Cyclodextrin-17-OH DHA Complex

There is provided a second novel process for concentrating 17-OH DHA from cow's milk. This method begins with the widely-available milk fat fraction that is a byproduct of the production of skim milk. In other embodiments, the starting fluid is a marine oil such as fish oil, krill oil or algae oil. In other embodiments, the starting fluid is an algae-derived oil. Next, it is observed that the melting points of long chain fatty acids are substantially controlled by the number of cis-double bonds in the molecule, as shown in Table IV below. Because 17-OH DHA has five double bonds, it likely has one of the lowest melting points of the fatty acids in milk fat. This feature can be exploited to concentrate 17-OH DHA in milk fat. Accordingly, in one embodiment, the milk fat fraction is subject to selective freezing in a temperature range of about at −20° C. to −40° C., thereby separating the lowest melting point molecules (i.e., those fatty acids having 4-6 cis bonds) from the remainder of the fat fraction. It is believed that this step removes about 95% of the fatty components in the milk fat fraction, and so concentrates 17-OH DHA by a factor of about 19.

TABLE IV
(Weiss, Lipids Health Disease, 2013, 12, 89 Tables 1-3)

| | Cis Double | | Melting Point |
|---|---|---|---|
| Constitutent | bonds | Concentration | (° C.) |
| Palmitic Acid | 0 | 23.96% | 64 |
| Stearic Acid | 0 | 6.91% | 70 |
| Oleic Acid | 1 | 46.29% | 13 |
| Palmitoleic Acid | 1 | 3.35% | 1 |
| Linoleic Acid | 2 | 14.42% | −5 |
| Gamma-Linolenic | 3 | 0.09% | −11 |
| Acid | | | |
| Alpha-linolenic Acid | 3 | 1.07% | −12 |
| Arachidonic Acid | 4 | 2.09% | −49 |
| Docosahexaenoic | 6 | 1.15% | −44 |
| Acid | | | |
| Eicosapentaenoic | 5 | 0.08% | −65 |
| Acid | | | |
| Lipoxin A4 | 1 | 15.55 ng/ml | ? |
| Resolvin E1 | 2 | 4.24 ng/ml | ? |
| Resolvin D1 | 3 | 9.42 ng/ml | ? |
| 17-OH DHA | 5 | 53.38 ng/ml | ? |

Next, we make the observation that monohydroxyfatty species are rapidly taken up by cells and esterified into triglycerides (Stenson, Prostaglandins, 1983, August, 26(2) 253-64, and Brezinski, PNAS USA, 1990 August, 87(16) 6248-52), and because adipose cells are extensively present in mammaries, it may reasonably be concluded that the fatty acids in breastmilk are also present in the triglyceride form. We then observe that hydroxyfatty acids can be selectively released from triglycerides by PLA2 without releasing the nonhydroxylated fatty acid species from the triglycerides (van Kuijk, Trends in Biochem. Sci., 12, 1987, 31-34; Bayon, Plant Physiology, April 2015, 167, 1259-70; and Bafor, Biochem. J., 1991, 280, 507-514). Accordingly, we can selectively release hydroxyfatty acids from its parent triglyceride by contacting the low MP milk fat fraction against immobilized PLA2.

Once the hydroxyfatty acids (and 17-OH DHA in particular) are present in their free form, they can be selectively removed from solution by contacting the solution with a cyclodextrin. Beta-Cyclodextrin is a lipophilic tube having an inner pore size of about 7 Angstroms (U.S. Pat. No. 4,902,788), and so they can be used to remove/concentrate lipophilic molecules having a size less than 7 Angstroms. Because the cis bond-driven folding of DHA causes it to have a radial dimension of about 5 Angstroms (Yonezawa, Int. J. Mol. Med., 2006, October, 18(4) 583-8 at Table 1), and 17-OH DHA is structurally quite similar, it is reasonable to conclude that 17-OH DHA has a radial dimension of about 7 Angstroms and so can likewise enter into and thereby be concentrated in cyclodextrins. Moreover, the nonhydroxylated fatty acids, such as DHA, remain in triglyceride form. Because there are 3 DHA molecules in a DHA triglyceride the dimensions of the triglyceride is probably about 15 Angstroms, and so would be much too large to be captured by beta-cyclodextrin. Accordingly, the cyclodextrins can selectively concentrate free 17-OH DHA from triglycerides.

In one embodiment, the cyclodextrin is present as a cyclodextrin carbonate nanoparticle, as described in Zhang, Intl. J. Nanomedicine, 2015, 10, 3291-3302. It is believed that both the cyclodextrin and carbonate components are perfectly safe for infants. Because Zhang's cyclodextrin-carbonate nanoparticle has a pore size of about 136-242 Angstroms, 17-OH DHA (which has a 5 Angstrom dimension) can easily diffuse through its pore system to be ultimately capture by the cyclodextrin. Moreover, cyclodextrin carbonate nanoparticles can be used to build a filter column through which the free hydroxylated fatty acid solution can be passed. Once in the cyclodextrin carbonate nanoparticle column, the free hydroxylated fatty acids (including 17-OH DHA) will enter the pore of the cyclodextrin and be captured thereby.

In another embodiment, the cyclodextrin is replaced by zeolite. Zeolite has been ingested for centuries by pregnant women in the form of clay to capture the nutritional mineral content of clay, relieve vomiting and nausea, and protect the digestive tract. Zeolite has a pore size of about 10 Angstroms (Du, J. Physics Chem. Solids, 68(2007) 1692-99), and so they can be used to remove/concentrate molecules having a size less than 10 Angstroms. Because the cis bond driven folding of DHA causes it to have a radial dimension of about 5 Angstroms (Yonezawa, Int. J. Mol. Med., 2006, October, 18(4) 583-8 at Table 1), and 17-OH DHA is structurally quite similar, it is reasonable to conclude that 17-OH DHA can likewise be concentrated in zeolite.

In another embodiment, the cyclodextrin is replaced by mesoporous silica. Mesoporous materials have a pore size of 20-500 angstroms (Wikipedia), and so they can be used to remove/concentrate lipophilic molecules having a size less than50-300 angstroms. Because DHA has a 5 angstrom size, and 17-OH DHA is structurally quite similar, it is reasonable to conclude that 17-OH DHA can likewise be concentrated in mesoporous silica.

In another embodiment, the cyclodextrin is replaced by octadecyl silyl silica (OSS). OSS has been used to selectively remove/concentrate hydroxylated molecules (i.e., prostaglandins) from phospholipids (Powell, Prostaglandins, 1980, November, 20(5) 947-57). Because 7-OH DHA is likewise hydroxylated, it is reasonable to conclude that 17-OH DHA can likewise be concentrated in OSS.

In sum, a 17-OH DHA concentrated fraction of milk can be made by:
a) Removing the fat fraction from milk,
b) Freezing the fat fraction at about −30° C. to separate out the low melting point fatty acids from the higher melting point fatty acids,
c) Contacting the low melting point molecules with immobilized PLA2 to selectively free the hydroxyfatty acids from the triglycerides, and
d) Contacting the free hydroxyfatty acids with cyclodextrins (or a substitute described above) to selectively capture the free hydroxyfatty acids without capturing the triglycerides.

The 17-OH DHA/cyclodextrin complex can then be orally administered to the patient, wherein the 17-OH DHA is slowly released by the cyclodextrin.

Therefore, there is provided a method of making a concentrated hydroxyfatty acid fraction from a DHA-containing fluid comprising i) low melting point fatty acids and ii) high melting point fatty acids, wherein both acids are present in triglycerides, comprising the steps of:
a) freezing the fluid to separate out the low melting point fatty acids from the high melting point fatty acids contained therein and thereby produce a low melting point-enriched fluid comprising parent triglycerides,
b) ex vivo contacting the low melting point-enriched fluid with PLA2 to selectively free hydroxyfatty acids from their triglycerides to produce a free hydroxyfatty acid-enriched fluid, and
c) ex vivo contacting the free hydroxyfatty acid-enriched fluid with a concentrator to selectively capture the freed hydroxyfatty acids within the concentrator to produce a concentrator having hydroxyfatty acid adsorbed thereon.

Preferably, the freezing step is carried out at about −20° C. to −40° C. The starting fluid can be selected from the group consisting of a marine oil, an algae oil, and a milk. In some embodiments, a step of separating out a fat fraction of the milk is carried out prior to step a), and step b) is carried out on the separated fat fraction.

In some embodiments, the concentrator is present as a cyclodextrin carbonate nanoparticle. In others, the concentrator is selected from the group consisting of cyclodextrin, zeolite, mesoporous silica, and octadecyl silyl silica (OSS).

In some embodiments, The method further comprises the step of:
d) administering the concentrator having hydroxyfatty acid adsorbed thereon to a human.

In some embodiments, the concentrator having hydroxyfatty acid adsorbed thereon is enriched in adsorbed 17-OH DHA.

In some embodiments, the concentrator is a solid porous body, and the hydroxyfatty acid is adsorbed within the porosity of the concentrator.

In some embodiments, the PLA2 is immobilized PLA2.

In some embodiments, the method further comprises the steps of:
d) releasing the hydroxyfatty acid from the concentrator to produce a hydroxyfatty acid-enriched solution, and
e) administering the hydroxyfatty acid-enriched solution to a human.

There is also provided a method of making a concentrated hydroxyfatty acid fraction from a DHA-containing fluid comprising triglycerides comprising hydroxyfatty acids, comprising the steps of:
a) ex vivo contacting the fluid with PLA2 to selectively free hydroxyfatty acids from their triglycerides and thereby produce a free hydroxyfatty acid-enriched fluid, and
b) ex vivo contacting the free hydroxyfatty acid-enriched fluid with a concentrator to selectively capture the freed hydroxyfatty acids within the concentrator to produce a concentrator having hydroxyfatty acid adsorbed thereon.

This method may further comprise the step of:
c) administering the concentrator having hydroxyfatty acid adsorbed thereon to a human.

This method may alternatively further comprise the steps of:
c) releasing the hydroxyfatty acid from the concentrator to produce a hydroxyfatty acid-enriched solution, and
d) administering the hydroxyfatty acid-enriched solution to a human.

In some embodiments, the concentrator having hydroxyfatty acid adsorbed thereon is selectively removed from the free hydroxyfatty acid-enriched fluid.

In some embodiments, the method may further comprise separating the concentrator having hydroxyfatty acid adsorbed thereon from the fluid.

In some embodiments, the method further comprises the step of:
c) administering the separated concentrator having hydroxyfatty acid adsorbed thereon to a human, wherein the administration is carried out once a day.

There is also provided a method of making a concentrated hydroxyfatty acid fraction from a DHA-containing fluid comprising esters comprising hydroxyfatty acids, comprising the steps of:
a) ex vivo contacting the fluid with an enzyme to selectively free hydroxyfatty acids from their esters and thereby produce a free hydroxyfatty acid-enriched fluid, and
b) ex vivo contacting the free hydroxyfatty acid-enriched fluid with a concentrator to selectively capture the freed hydroxyfatty acids within the concentrator to produce a concentrator having hydroxyfatty acid adsorbed thereon.

Chelated Magnolol Complex

Magnolol is an isomer of honokiol and is also derived from magnolia bark. In addition, magnolol is likewise a GABA delta agonist, increasing GABA currents by about 900% at a concentration of 10 uM (Alexeev, Neuropharmacology, 2012 June, 62(8), 2507-2514). Therefore, magnolol is deemed to be a suitable replacement for honokiol in the honokiol/ellagic acid assembly discussed above.

Further, examination of the magnolol structure reveals another interesting characteristic of magnolol. In particular, it has been observed that if the phenyl rings of magnolol are properly rotated, the two hydroxyl groups situated on the different rings approach each other. When the hydroxyls are in this “close approach” conformation, it is believed that magnolol can form a chelated complex with metal ions.

The chelated magnolol complex is interesting from a number of viewpoints. First, as opposed to the poor water solubility of pure magnolol, the chelated magnolol complex is likely highly water soluble. The high water solubility of the chelated magnolol complex facilitates the ability of magnolol to approach the epithelial cells in the GI tract by providing high dispersability, and thus increasing its bioavailability in oral administration.

Chelated 7,8 Dihydroxyflavone Complex

7,8 dihydroxyflavone (7,8 DHF) is a natural flavonoid found in Godmania aesculifolia, Tridax procumbens, and primula tree leaves. It is also available as supplement. Liu reported that 7,8-DHF (which is orally available and BBB penetrable) can specifically activate TrkB receptors (at a low concentration of 250 nM) and its downstream PI3K/Akt and MAPK receptors in the mouse hippocampus. 7,8-DHF can protect neurons from excitotoxic and oxidative stress-induced apoptosis and cell death. Moreover, 7,8-DHF promotes the survival and reduces apoptosis in cortical neurons of traumatic brain injury. Liu, Trans. Neurodegener. 2016; 5: 2. Accordingly, 7,8 DHF is of interest as an antidepressant.

Inspection of the 7,8 DHF molecule reveals adjacent hydroxyl groups attached to an aromatic ring. It is known that such structures have the ability to form chelation complexes with metals.

It is believed that a chelate complex of 7,8 DHF will have high water solubility, thus allowing for its uniform dispersal in the aqueous phase of the gastrointestinal tract, and thereby increasing its bioavailability. Once the well-dispersed 7,8 DHF chelate complex enters the stomach, the high acid content therein will release the metal ion from its complex with 7,8 DHF, leaving a well dispersed, free 7,8 DHF in solution.

Also according to Liu, it is further known that 7,8,2′ DHF and 7,8,3′ trihydroxyflavones (THF) are also potent Trkb agonists, increasing Akt phosphorylation over 150%. Liu, J. Med. Chem., Dec. 9, 2010; 53(23):8274-86. Because these molecules also possess the structure of adjacent hydroxyl groups attached to an aromatic ring, it is likewise believed that such structures have the ability to form chelation complexes with metals. Therefore, it believed that a chelate complex of 7,8 DHF; 7,8,2′ THF or 7,8,3′ THF (along with other TrkB-active hydroxyflavones having hydroxyls in both the 6 and 7 positions) will have high water solubility, thus allowing for its uniform dispersal in the aqueous phase of the gastrointestinal tract, and thereby increasing its bioavailability.

Once the well-dispersed hydroxyflavonoid chelate complex enters the stomach, the high acid content therein will release the metal ion from its complex with hydroxyflavonoid, leaving a well dispersed, free hydroxyflavonoid in solution.

As these hydroxyflavones having hydroxyls in either the 6,7 or 7,8 positions have been shown to be Trkb agonists, and TrkB is the prime receptor for BDNF, it is believed that these phytochemicals would be good treatment candidates for mothers diagnosed with PND who have low serum BDNF levels, as the phytochemical would serve to augment the heretofore insufficient serum BDNF level in activating the TrkB receptor. Moreover, it has been reported that there is an association between low serum BDNF levels in early pregnancy and antenatal depression. Fung, BMC Psychiatry, 2015 March 10, 15,43. Therefore, it is believed that these chelates would be good candidates for mothers who have low serum BDNF levels in early pregnancy, so as to prevent the onset of antenatal depression in these mothers.

A. 7,8 Dihydroxyflavone/Hesperetin Glucuronide Mixed Self-Assembly

Hesperidin is a natural flavonoid glycoside found in citrus. Its deglycosylated flavonoid metabolite (hesperetin) is commonly found in significant quantities (up to micromolar levels) in human mother's milk. Song, Nutrition, 2013 January; 29(1):195-202 Hesperidin administration is further known to increase BDNF levels in chronically-depressed rats (Donato, Brain Res. Bull., 2014 May; 104:19-26). Moreover, its metabolite hesperetin induces BDNF (Hwang, J. Agric. Food Chem., May 25, 2011; 59(10):5779-85). Another metabolite, hesperetin glucuronide, has a log P of 0.12 (Chemspider), and so, it is reasonably assumed that hesperetin glucuronide will behave like a surfactant, and thereby have the ability to form micelles, liposomes and MLVs.

It is further observed that the flavanone hesperetin glucuronide shares the same basic flavonoid structure (with the exception of a double bond) as the flavonol 7,8 DHF. Therefore, it is believed that 7,8 DHF might intercalate within a hesperetin glucuronide self-assembly to form a mixed structure that will slowly release 7,8 DHF therefrom.

This same hesperetin glucuronide molecule has been found to activate PPAR-gamma receptors (EC˜100 uM) and so likely has utility as an anti-diabetic agent. Gamo, Chem. Pharm. Bull., 62(5), 491-493 (2014). Therefore, it is believed that this 7,8 Dihydroxyflavone/Hesperetin Glucuronide mixed self-assembly can be useful for treating the Group 4a phenotype described above.

B. Hesperetin/Hesperetin Glucuronide Mixed Self-Assembly

As discussed above, it is known that hesperetin is found in significant quantities in mother's milk and is known to induce BDNF. Therefore, it is an attractive candidate for use as an antidepressant in PND. However, its high lipophilicity (Log P=2.90) causes it to have a low dispersability in water and therefore a low bioavailability. It is further observed that hesperetin shares the identical flavonoid structure as its metabolite, hesperetin glucuronide. As discussed above, the amphiphilic log P of hesperetin glucuronide leads one to believe that it can self-assemble. Therefore, it is believed that hesperetin might intercalate within a hesperetin glucuronide self-assembly to form a mixed structure that will be well dispersed in water (and therefore have a high bioavailability) and will also likely provide for slow release of hesperetin therefrom.

C. Pinocembrin/Hesperidin Glucuronide Mixed Self-Assembly

Pinocembrin, the primary flavonoid in Swiss honey, has been reported to suppress apoptosis in neurons with an EC₅₀ of only 100 nM. Jang, PNAS, 107, 6, 2687-92. It is further observed that hesperidin glucuronide shares the identical flavanone structure as pinocembrin. Therefore, it is believed that pinocembrin can intercalate within a hesperidin glucuronide self-assembly to form a mixed structure that will slowly release pinocembrin therefrom.

Likewise, owing to their structural similarity, it is believed that pinocembrin can intercalate within a saponin self-assembly (such as a soyasaponin self-assembly) to form a mixed structure that will slowly release pinocembrin therefrom.

D. 7,8 Dihydroxyflavone/7,8 Dihydroxyflavone Glucuronide Mixed Self-Assembly

As discussed above, 7,8 dihydroxyflavone is an attractive candidate as a PND anti-depressant because it is a potent TrkB agonist. It is now observed that its metabolite, 7,8 dihydroxyflavone glucuronide, shares common structure with hesperetin glucuronide and so likely has an amphiphilic log P similar to hesperetin glucuronide. This amphiphilic property would make 7,8 dihydroxyflavone glucuronide a good candidate for self-assembly.

It is further observed that 7,8 dihydroxyflavone glucuronide shares the identical flavonoid structure as 7,8 dihydroxyflavone. Therefore, it is believed that 7,8 dihydroxyflavone can intercalate within a 7,8 dihydroxyflavone glucuronide self-assembly to form a mixed structure that will provide for high bioavailability of 7,8 dihydroxyflavone and slowly release 7,8 dihydroxyflavone therefrom.

As with the above chelates, it is believed that these self-assemblies that augment BDNF or activate TrkB would be good candidates for expectant mothers who have low serum BDNF levels in early pregnancy, so as to prevent the onset of antenatal depression in these mothers.

E. Hyperoside/G-Rutin (SJW) Mixed Self-Assembly

St. John's Wort (SJW) is one of the few antidepressant preparations available to a nursing mother diagnosed with PND for which there is evidence demonstrating both safety and efficacy. It has been reported that in vivo rat experiments demonstrate that the hyperoside (0.6 mg/kg) and quercetin-3-glucuronide (0.6 mg/kg) constituents in SJW appear (along with hyperforin) to be the entities responsible for the anti-depressant effect of SJW, and that they work by reducing the HPA axis function by reducing plasma levels of ACTH and cortisol by 40-70% (Butterweck, Planta Med., 2000, February, 66(1) 3-6). As there are some concerns with SJW and its side effects, in particular a deleterious interaction between the hyperforin in SJW and blood pressure medication (Narhstedt, J. Nat. Prod., May 2010, 73(5) 1015-21), it would appear that providing just the beneficial components of SJW to the PND mother seems like an attractive option. In this respect, hyperoside in particular seems to have promise as a PND treatment option, as it has been shown to elevate BDNF transcription in PC12 cells (Zheng, Phytomedicine, Jan. 15, 2012, 19(2) 145-9). Moreover, a hyperoside-rich preparation (Venetron) is commercially available in the US and has been clinically demonstrated to improve symptoms of depression. Maypro, “Venetron: A Promising New and Efficacious Dietary Ingredient for mood support and sleep.”

However, it is further observed that hyperoside is rather lipophilic, possessing a log P of 1.75 (Chemspider). Because of this high lipophilicity, hyperoside is likely not very water soluble and so likely has difficulty in attaining a high oral bioavailability. Moreover, being a polyhydroxyflavone, hyperoside is likely subject to severe first-pass metabolism, thereby reducing its potency. Accordingly, it is a goal to provide hyperoside in a delivery package that increases its bioavailability and release profile.

In order to overcome this oral bioavailability issue, it is proposed that hyperoside be housed in a self-assembly of glycosylated rutin (G-rutin). G-rutin is a natural amphiphilic phytochemical that is found in buckwheat and the Japanese Pagoda tree (Morita, Cereal Chem., 1996, 73(1) 99-104). It is currently marketed in the US as an active ingredient in Eucerin^{R} skin lotions, and has particular interest for consumers with sun allergy. Of interest, G-rutin has been reported to self-assemble into micellar aggregates. Tozuka, Eur. J. Pharm. Biopharm., 2012 September; 82(1):120-6. These micelles should have a high water solubility, and so should have a high oral bioavailability.

It is further appreciated that hyperoside and G-rutin have a special relationship by virtue of their nearly identical structures. In particular, each of hyperoside and G-rutin has a quercetin-based lipophilic portion and glucose moieties attaching off the same 3-OH of the base quercetin molecule.

Because of the near identity in their chemical configurations, it is believed that hyperoside will neatly intercalcalate itself within the G-rutin superstructure of the micelle. This neat intercalation will result in enhanced bonding between the lipophilic portions of the quercetin base molecules, between the hydroxyls of the base quercetin molecules, and between the glucose structures that attach to the 3-OH portion of the quercetin molecules. This enhanced bonding will likely result in an extended time of release of the hyperoside from the self-assembly, which may allow for a much longer hyperoside half-life in circulation, thereby increasing the bioavailability of hyperoside. Accordingly, it may be appropriate to consider the hyperoside/G-rutin mixed self-assembly as a phytosome.

In some embodiments, the hyperoside/G-rutin self-assembly manifests itself as a micelle. In others, the hyperoside/G-rutin mixed self-assembly manifests itself as a liposome. In others, the hyperoside/G-rutin self-assembly manifests itself as a multi-lamellar vesicle (MLV).

F. Q3G/G-Rutin Mixed Self-Assembly

It is further observed that Q3G is rather lipophilic, possessing a log P of 2.10 (Chemspider). Because of this high lipoiphilicity, Q3G is not very water soluble and so likely has difficulty in attaining a high oral bioavailability. Moreover, the plasma elimination half-life of Q3G has been reported to be only 2.33 hours (Mullen, Br. J. Nutr. 2006 July; 96(1):107-16.) Mullen further reported that the profile of metabolites excreted in urine was markedly different to that of plasma with many of the major urinary components, including quercetin-3′-glucuronide, two quercetin glucoside sulphates and a methylquercetin diglucuronide, absent or present in only trace amounts in the bloodstream, indicative of substantial phase II metabolism.

Therefore, it is a goal of the present invention to increase the bioavailability and half-life of Q3G.

In order to overcome this oral bioavailability issue, it is proposed that Q3G be housed in a self-assembly of glycosylated rutin (G-rutin). G-rutin is an amphiphilic molecule that has been reported to self-assemble into micellar aggregates. Tozuka, Eur. J. Pharm. Biopharm., 2012 September; 82(1):120-6. These micelles should have a high water solubility, and so should have a high oral bioavailability. Therefore, G-rutin should be able to sufficiently deliver Q3G from the GI tract into the circulatory system.

It is further appreciated that Q3G and G-rutin have a special relationship by virtue of their highly similar structures. In particular, each of Q3G and G-rutin has a quercetin-based lipophilic portion and a glucose-like portion attaching off the same 3-OH of the base quercetin molecule.

Because of the near identity in their chemical configurations, it is believed that Q3G will neatly intercalcalate itself within the G-rutin superstructure of the micelle. This neat intercalation will result in enhanced bonding between the lipophilic portions of the quercetin base molecules, between the hydroxyls of the base quercetin molecules, and between the glucose-like structures that attach to the 3-OH portion of the quercetin molecules. This enhanced bonding will likely result in an extended time of release of the Q3G from the self-assembly, which may allow for a much longer Q3G half-life in circulation, thereby increasing the bioavailability of Q3G. Accordingly, it may be appropriate to consider the Q3G/G-rutin assembly as a phytosome.

In some embodiments, the G-rutin/Q3G self-assembly manifests itself as a micelle. In others, the G-rutin/Q3G self-assembly manifests itself as a liposome. In others, the G-rutin/Q3G self-assembly manifests itself as a multi-lamellar vesicle (MLV).

As both hyperoside and Q3G appear to promote antidepressant state by reducing ACTH and cortisol levels (see Butterweck above), and high cortisol levels are implicated in the phenotype 3 presented above, it appears that the hyperoside/G-rutin self-assembly and the Q3G/G-rutin self-assembly presented herein would have special applicability to the mother diagnosed with the above Type 3 phenotype.

G. Ginsensoide Liposomes and MLVs

The ginsenoside Rg3 has been reported to exert antidepressant effects in several animal models (Cui, J. Psychopharmacol., 2012 May; 26(5) 697-713). Investigators have further reported the ginsenoside Rg2 reverses stress-induced depression-like behaviours and BDNF expression within the prefrontal cortex (Zhu X, Eur J Neurosci. 2016 July; 44(2):1878-85); the beneficial effects of ginsenoside Rg1 on chronic stress-induced depression-like behaviours, BDNF expression and phosphorylation of PKA and CREB, (Liu Z, Neuroscience. May 13, 2016; 322:358-69); the beneficial effect of ginsenoside Re on depression and anxiety-like behaviours induced by repeated immobilization (Lee B, J Microbiol Biotechnol. 2012 May; 22(5):708-20); and the antidepressant effects of ginsenoside Rg1 due to activation of BDNF signalingand neurogenesis in the hippocampus (Jiang, Br J Pharmacol. 2012 July; 166(6):1872-87).

Therefore, in some embodiments, ginsenoside self-assemblies (and in particular liposomes and MLVs) are administered to the mother diagnosed with either antenatal or postnatal PND.

As discussed above, it has been reported that ginsenosides self-assemble into micelles, and that self-assembled ginsenoside micelles can be tuned to have release rates from days to months. Xiong, Int. J. Pharm. Aug. 6, 2008; 360(1-2):191-6. The tuning is performed by varying the concentration of the ginsenoside in the initial solution, with higher concentrations leading to slower release rates. Xiong teaches that loading determines release rate. In some embodiments, the loading of the ginsenoside self-assembly (and in particular Rg3) is targeted to provide a release rate that corresponds to essentially complete release in about 24 hours and a 50% release rate at about 12 hours. This loading and corresponding rate would enable the mother to take only one dose a day (and thereby promote compliance more than a multiple-dose-per-day routine) while still enabling a habit-forming routine of taking one dose per day (thereby promote compliance more than a one-dose-every-few-days routine).

In some embodiments, the ginsenoside self-assembly (and in particular, the Rg3 self-assembly) is presented in the form of liposomes. It is believed that the liposomal form provides an advantage during pulmonary administration of the the ginsenoside self-assembly (and in particular, the Rg3 self-assembly). Liposomes are generally on the order of 100-200 nanometers (and so are categorized as fine particles), while micelles are much smaller at about 10-20 nm (and so are categorized as ultrafine particles). Because a substantially larger fraction of micelles are exhaled after pulmonary administration, liposomes provide an advantage (over micelles) in that their relatively larger size provides a much more efficient pulmonary administration. Liposomes can also deliver hydrophilic molecules housed in their aqueous cores.

In some embodiments, the ginsenoside self-assembly (and in particular, the Rg3 self-assembly) is presented in the form of multi-lamellar vesicles (MLVs). It is believed that the MLV form provides an advantage during pulmonary administration of the ginsenoside self-assembly (and in particular, the Rg3 self-assembly). MLVs can be made to a size on the order of a few microns. Because a substantially larger fraction of micelles and liposomes are exhaled after pulmonary administration, MLVs provide an advantage (over micelles and liposomes) in that their relatively larger size provides a much more efficient pulmonary administration. Thus, MLVs have the particle diameter in the range needed for aerosol delivery to the alveolar region. Zaru, Eur. J. Pharmaceutics Biopharm., 67(2007) 655-666 at 663. These ginsenoside inventions will have a slower release than conventional drug-loaded liposomes because the drug forms part of the superstructure of the micelle/liposome/MLV and so has enhanced intermolecular bonding.

In sum, the above disclosure provided a number of broad concepts involving matching self-assembling amphiphilics with active agents capable of intercalating within the self-assembly. Some of these examples are listed in Table V below:

TABLE V

| | | | Amphiphilic |
|---|---|---|---|
| Active Class | Active Example | Amphiphilic Class | Example |
| flavone | 7,8 dihydroxyflavone | Flavone-7-O- | 7,8 dihydroxyflavone- |
| | | glucuronide | 7-O-glucuronide |
| Flavone-3-O- | Miquelianin; | Flavone-3-O- | G-Rutin |
| glucuronide | | glycoside | |
| Flavone-3-O- | hyperoside | Flavone-3-O- | G-Rutin |
| glycoside | | glycoside | |
| Flavanone | Hesperetin; pinocembrin | Saponin | soyasaponin; quillaja |
| Flavone | 7,8 dihydroxyflavone | Flavone-3-O- | G-Rutin |
| | | glycoside | |
| Isoflavone | equol | saponin | soyasaponin; quillaja |
| isoflavone | equol | Isoflavone glycoside | diadzin |
| Steroid | allopregnanolone | saponin | soyasaponin; quillaja |
| Flavanone | Hesperetin; pinocembrin | Flavanone-7-O- | Hesperetin-7-O- |
| | | glucuronide | glucuronide |

Thus, generally, there is provided herein a mixed self-assembly comprising:
a) a hydroxylated flavonoid-7-O-glucuronide self-assembly (preferably a hydroxylated flavanone-7-O-glucuronide self-assembly), and
b) a hydroxylated flavonoid (preferably a hydroxylated flavanone) intercalated within the self-assembly.

Thus, generally, there is provided herein a mixed self-assembly comprising:
a) a hydroxylated flavonoid-3-O-glycosyl self-assembly (preferably a hydroxylated flavonol-3-O-glycosyl self-assembly, and
b) a hydroxylated flavonoid (preferably a hydroxylated flavonol) intercalated within the self-assembly.

Thus, generally, there is provided herein a hydroxyflavone having adjacent hydroxyl groups that is chelated by a metal (preferably zinc) that forms a complex with the two hydroxyls. Preferably, the hydroxyflavone chelate forms a self-assembly with other similar complexes.

Baicalin

It is believed that sufficient progesterone is important to the expectant mother in two ways. First, it has been reported that lower progesterone in the second trimester of pregnancy is associated with greater negative emotional responses to stress in that trimester. Crowley, Psychopharmacology, 2016 April, 233(7), 1299-310. Second, it has been repeatedly reported that prophylactic administration of progesterone can reduce the incidence of preterm births (Saccone, Ultrasound Obstet Gynecol., Aug. 22, 2016). It is believed that preterm birth is positively associated with symptoms of PND. For example, mothers of early, moderate, and late preterm infants reported similar rates of possible depression (20%, 22%, and 18%, respectively) one month after NICU discharge (Hawes, J. Pediatr. Aug. 5, 2016. pii: S0022-3476(16)30531-5). These depression rates associated with preterm births are somewhat higher than the 10-15% PPD rate generally reported. Moreover, another investigator group reported that premature infants at three months exhibit more withdrawal behavior and their mothers reported elevated maternal depressive symptoms as compared with the full-born group. At 12 months, the mothers of the premature infants reported more child internalizing behavior (Moe, Infant Behay. Dev., 2016 Augist; 44:159-68). Therefore, it appears that properly provided progesterone can be helpful to the expectant mother having low progesterone levels or at risk for delivering preterm. However, progesterone has a relatively short circulatory half-life. Anand Kumar. Proc. Nat. Acad. Sci. USA, 1982 July; 79(13):4185-9. Therefore, therapies that help increase the circulatory half-life of progesterone may be useful.

Baicalin is a glucuronidated flavonoid found in the Chinese Skullcap extract, which has been used in Chinese medicine for miscarriage and threatened abortion. Chen, Evidence-based Compl. Alter. Med., Volume 2011, 408714. Baicalin can exert and antiabortive effects by reducing IFN-gamma levels and elevating progesterone. Ma, Am. J. Chin. Med., 2009, 37(1) 85-95 and Chen, J. Steroid Biochem. Mol. Biol. 2015 May; 149:11-6 (Baicalin elevating progesterone). In an in vitro study of the effect of baicalin on deidua cells, baicalin showed a nonsignificant trend in elevating progesterone (Wang, J. Immunol. Research, Vol. 2014, 859812, FIG. 6). Baicalin has demonstrated tocolytic properties, meaning it can delay labor, and investigators attribute the tocolytic properties of baicalin to its ability to increase progesterone (Chen, J. Steroid Biochem. Mol. Biol. 2015 May; 149:11-6). Baicalin appears to be a better tocolytic agent than its aglycone bacailein. Chen, citing Ma, Chin. J. Vet. Sci. 27 (2007) 412-415 (in Chinese).

Therefore, without wishing to be tied to a theory, it appears that baicalin administration to an expectant mother might be useful for increasing the mother's progesterone levels, and thereby elevating mood and decreasing the risk of preterm birth.

It is further noted that bacailin is structurally similar to scutellarin (differing by a single hydroxyl), in that each is a hydroxylated flavonoid -7-O-glucuronide. Accordingly, it is reasonable to expect that their pharmacologic profile should be reasonably similar.

It appears that hydroxylated flavonoid-7-O-glucuronides such as scutellarin are poorly orally available, demonstrating a bioavailability of less than 3% (Liu, Eur. J. Pharm. Biopharm., 2008 November, 70(30 845-52. Because of the structural similarity of scutellarin and baicalin, it is reasonable to conclude that baicalin has a similarly poor oral bioavailability. Liu goes on to report, however, that providing spray-dried scutellarin nanoparticles through a pulmonary administration increased the bioavailability of the scutellarin to about 77%, and that adding a mucoadhesive excipient to the formulation increased to bioavailability to over 95%.

Because bacailin is structurally similar to scutellarin, it is reasonable to conclude that likewise providing spray-dried baicalin nanoparticles through a pulmonary administration may increase its bioavailability from a level of less than 3% to about 77%, and that adding a mucoadhesive excipient to the formulation might increase its bioavailability to over 95%.

Therefore, there is provided a method of treating an expectant mother (preferably having a risk of preterm birth), comprising the steps of:
a) providing an inhaler housing a formulation comprising spray-dried baicalin nanoparticles and a mucoadhesive excipient,
b) carrying out a pulmonary administration of the formulation to the expectant mother.

Liu, supra, reported that the plasma concentration of scutellarin in their experiments decreased by a factor of about 10 over the course of about an hour, thereby suggesting that scutellarin (and by implication other hydroxylated flavonoid-7-O-glucuronides such as baicalin) has a short circulatory half-life. It has been reported that baicalin has a log P of 1.27 (Liang, J. Agric. Food Chem., Aug. 12, 2009; 57(15):7118-24) and so can be considered to be amphiphilic. Therefore, in preferred embodiments, this amphiphilic quality is exploited to provide baicalin in the form of a self-assembly such as a liposome or MLV that can provided for an extended release of baicalin.

Cerebroside and bc1-2

In some embodiments, an amphipathic cerebroside is made into a self-assembly in the form of a liposome or MLV, and is delivered (preferably by the oral or pulmonary route) to a mother diagnosed with PND. Cerebrosides are endogenous molecules known to be present in human milk (Newburg, Lipids, 1992 November; 27(11):923-7). Certain aquatic cerebrosides have also been reported to dramatically increase the gene expression of B-cell lymphoma 2 (Bcl-2). Wu, J. Oleo Sci., 2013; 62(9):717-27. bcl-2 is an anti-apoptotic gene that has been implicated in mediating neuronal plasticity. Manji, Psychopharmacol. Bull., 2001 Spring; 35(2):5-49. Therefore, it is expected that administration of a cerebroside to a mother diagnosed with PND should increase her neuronal plasticity and thereby alleviate her symptoms of depression.

Cancer Applications

In addition to applications of phytochemical self-assemblies directed to maternal depression, it is believed these phytochemical self-assemblies can also be directed to certain forms of cancer.

A. Hyperoside and Q3Mixed Micelles

It has also been observed that both hyperoside and Q3G are beta-adrenergic antagonists. This quality is relevant because there have been at least six retrospective studies that have consistently demonstrated a connection between beta-blocker use (and beta-2 antagonist propranolol, in particular) and about a 50% reduction in the occurrence of metastatic breast cancer (and triple negative breast cancer (TNBC), in particular). See a) Melhem-Bertrandt, “Beta-blocker use is associated with improved relapse-free survival in patients with triple-negative breast cancer.” J Clin Oncol, 29: 2645-2652, 2011.; b) Barron, “Beta blockers and breast cancer mortality: A population-based study”, J Clin Oncol, 29: 2635-2644, 2011; c) Powe, “Beta-blocker drug therapy reduces secondary cancer formation in breast cancer and improves cancer specific survival”. Oncotarget, 1: 628-638, 2010; d) Botteri, “Therapeutic effect of β-blockers in triple-negative breast cancer postmenopausal women.” Breast Cancer Res. Treat. 2013 August; 140 (3):567-75, and e) Choy, “Inhibition of β2-adrenergic receptor reduces triple-negative breast cancer brain metastases: The potential benefit of perioperative β-blockade”, Oncology Reports, 35, 2016, 3135-42; f) Parada-Huerta, “Metastasis Risk Reduction Related with Beta-Blocker Treatment in Mexican Women with Breast Cancer”, Asian Pac. J. Cancer Prev., 2016; 17(6):2953-7.

In addition, beta-blockers also appear to increase the survival of ovarian cancer patients (Sood, “Clinical impact of selective and nonselective beta-blockers on survival in patients with ovarian cancer”, Cancer, 121: 2015). Currently, an interventional study is being conducted at the MD Anderson Cancer Center to examine the effect of a non-selective β-blocker plus standard chemotherapy (paclitaxel and carboplatin or possibly docetaxel) to treat ovarian cancer. “Feasability Study: Therapeutic Targeting of Stress Factors in Ovarian cancer Patients”. NCT01504126.

It has been reported that quercetin-3-O-glucuronide (Q3G), a metabolite of the phytochemical quercetin, is also a beta-adrenergic antagonist. Yamazaki, Arch. Biochem. Biophys. Sep. 1, 2014; 557:18-27. Yamazaki reported that Q3G (0.1 μM) suppressed invasion of MDA-MB-231 breast cancer cells (which are TNBC cells) and MMP-9 induction, and inhibited the binding of [(3)H]-NA to β2-AR. Yamazaki concluded that Q3G may function to suppress invasion of breast cancer cells by controlling β2-adrenergic signaling, and may be a dietary chemopreventive factor for stress-related breast cancer. Based upon its behavior as a beta-adrenergic antagonist and its exemplary safety profile that is essentially free of side effects, it is proposed herein to use Q3G and/or hyperoside in the self-assemblies described above as a chemotherapeutic for TNBC and ovarian cancers in patients already diagnosed with these cancers in order to prevent metastatic breast or ovarian cancer.

B. Propranolol Liposomes and MLVs

It has also been further been observed the positive results in the above-mentioned cancer epidemiology studies concerning beta-blockers appear to correlate strongly with the specific use of propranolol. Although propranolol has been successfully used to treat hypertension and is on the WHO List of Essential Medicines, it nonetheless has some drawbacks in that its relatively rapid metabolism often requires that it be taken 2-4 times daily and its extent of metabolism is inconsistent across different patients, thereby requiring a lengthy tritation procedure at the beginning of the therapy. Therefore, it is another goal to improve the metabolic and pharmacokinetic profile of propranolol.

To this end, it is observed that the literature has reported that propranolol is sufficiently amphiphilic as to form micelles, with a critical micelle concentration (CMC) of about 0.13 mol/L (Ruso, J. Chem. Eng. Data, 2003, 48 (6), pp 1597-1602). See also Schreier, Biochimica et Biophysica Acta, 1508 (2000) 210-234 for reports of propranolol micelles. This amphiphilic quality of propranolol makes it reasonable to expect that the skilled artisan can also make self-assembled propranolol structures including gels, multi-lamellar vesicles (MLVs) and liposomes. These complex self-assembled propranolol structures can then be used to treat TNBC and ovarian cancer patients and thereby prevent metastatic cancer.

C. Ginsenoside Liposomes and MLVs

It has further been reported that ginsenosides appear to be efficacious in treating cancers and especially lung cancer. In particular, one set of investigators has reported that administration of one particular ginsensoide (Rg3) to lung cancer patients has significantly increased the postoperative life span of those patents, and that Rg3 performed substantially as well as standard chemotherapy (Lu, Chin. J. Integr. Med., 2008 March. 14(1) 33-6). Lu further reported special efficacy of Rg3 against patients having a “positive VEGF expression” phenotype. Another set of investigators has reported that the combination of the ginsenoside Rg3 along with EGFR-TKI chemotherapy produced a 20% increase in the duration of progression free survival in lung cancer patients (Li, Oncotarget, Sep. 16, 2016). Therefore, in some embodiments, the complex self-assembled ginsenosides self-assemblies discussed above are administered to lung cancer patients, preferably through the pulmonary route.

D. Baicalin and COPD

Baicalin has also been demonstrated to be an inhibitor of prolyloligopeptidase (POP). Tarrago, Bioorg. Med. Chem., Aug. 1, 2008; 16(15):7516-24. Tarrago reported that baicalin inhibited prolyloligopeptidase in a dose-dependent manner, with inhibition experiments using baicalin analogs showing that the sugar moiety was not necessary for activity. The IC₅₀ of baicalin and its aglycone derivative baicalein were rather similar, showing that the sugar moiety was not involved in the interaction of baicalin with POP.

This anti-POP feature of baicalin may signal a utility of baicalin in preventing lung cancer, as it has been reported that cigarette smoke-induced lung emphysema in mice is associated with POP, an enzyme associated with collagen breakdown. Braber, Am J Physiol Lung Cell Mol Physiol. 2011 February; 300(2):L255-65. One clinical trial in which a POP inhibitor (Roflumilast) was provided to COPD patients reported reduced pulmonary inflammation through decreasing prolyl endopeptidase activity and AcPGP. The investigators correlated lower AcPGP levels with blunted markers of neutrophilic inflammation, and concluded that inhibiting this self-propagating pathway lessens the overall inflammatory burden, which may alter the natural history of COPD, including the risk of exacerbation. Wells, Am. J. Respir. Crit. Care Med., Oct. 15, 2015; 192(8):934-42 (NCT 01572948).

Therefore, there is provided a method of treating a patient with COPD (preferably a smoker with COPD), comprising the steps of:
a) providing an inhaler housing a formulation comprising spray-dried baicalin nanoparticles and a mucoadhesive excipient,
b) carrying out a pulmonary administration of the formulation to the patient.

Ganglioside-Containing Vesicles

In some embodiments, the self-assembled structures can be coated with a layer of a mucoadhesive (such as pectin) in order to enhance the binding of the self-assembly to the wall of the GI tract or lung.

Liposomes are often used to orally deliver drugs to the circulation. Ahmad, Curr Drug Metab., 2015; 16(8): 633-44. Oral lipsomes are typically made of amphiphilic lecithin, which contains a hydrophilic head group and hydrophobic tails. Lecithins are usually phospholipids, composed of phosphoric acid with choline, glycerol or fatty acids, usually glycolipids or triglyceride. Glycerophospholipids in lecithin include phosphatidylcholine, phosphatidyletahanolamine, phosphatidylinositol, phosphatidylserine and phosphatidic acid.

Although lecithin-based liposomes have been routinely used to orally deliver drugs, their use presents three challenges. First, lecithin-based liposomes do not robustly survive the acidity and bile present in the gastrointestinal tract. Taira, Drug Delivery, 11, 2; 123-128 (2004). Second, lecithin-based liposomes typically release their contents so quickly as to require multiple dosings per day for molecules with short half-lives. Third, lecithin-based liposomes that enter circulation are often susceptible to quick removal by RES uptake. Litzinger, Biochim. Biophys. Acta., Feb. 17, 1992, 1104(1)179-87.

Gangliosides are endogenous amphipathic molecules, and are present in human milk. Recently, gangliosides have been found to be highly important molecules in immunology. Natural and semisynthetic gangliosides are considered possible therapeutics for neurodegenerative disorders. See, for example, Mocchetti I (2005). “Exogenous gangliosides, neuronal plasticity and repair, and the neurotrophins”. Cell Mol Life Sci. 62 (19-20): 2283-94. Accordingly, orally-delivered gangliosides should be considered safe and even beneficial for mother and infant. Indeed, gangliosides have even been provided to 2230 children suffering from cerebral palsy, with the reported result of improved neurological symptoms. Xu, Chin. J. Clin. Rehab., 2005, 9, 122-123.

It has been recently found that adding gangliosides to the conventional lecithin-like liposome helps delivery of that active to the patient. First, they increase the robustness of the liposome towards the GI tract: “This study suggests that among the formulations used as oral drug carriers, those containing GM1 and GM type III have higher possibilities of surviving through the gastrointestinal tract.” Taira, Drug Delivery, 11, 2; 123-128 (2004). Second, ganglioside-containing lecithin liposomes that enter circulation are less susceptible to quick removal by RES uptake, thereby prolonging their lifetime in circulation. Chonn, J. Liposome Research, 2(3), 397-410, 1992. Third, adding gangliosides to the conventional liposomes can reduce the flux of glucose-6-phosphate (G6P) from the liposome into plasma to a level of about 5% per hour (see FIG. 2 of Taira), thereby allowing for nearly constant release of G6P from the liposome over the course of one day. Therefore, it is believed that gangliosides beneficially reduce the gaps in the liposome structure to reduce the flux of low molecular weight, hydrophilic molecules like G6P therethrough.

G6P has a molecular weight of about 260 daltons and a log P of −3.24. It is believed that other low MW molecules that are likewise hydrophilic should pass through a ganglioside-containing lecithin-based liposome with a comparable flux, thereby allowing for once a day dosing and a constant plasma concentration.

In particular, it is believed that molecules having a molecular weight of between about 100 and 400 daltons) and that are likewise hydrophilic (preferably a log P<0) should pass through a ganglioside-containing lecithin-based liposome with a comparable flux as G6P, thereby allowing for once a day dosing and a constant plasma concentration.

Preferably, it is believed that molecules having a molecular weight of between about 200 and 400 daltons, and between 200 and 300 daltons in some embodiments, and between 225 and 275 daltons in others) and that are likewise very hydrophilic (log P<−1) should pass through a ganglioside-containing lecithin-based liposome with a comparable flux as G6P, thereby allowing for once a day dosing and a constant plasma concentration.

More preferably, it is believed that molecules having a molecular weight of between about 100 and 400 daltons), have a cyclic component and are very hydrophilic (log P<0) should pass through a ganglioside-containing lecithin-based liposome with a comparable flux as G6P, thereby allowing for once a day dosing and a constant plasma concentration.

Taira's embodiments that displayed the desired flux used lecithin, cholesterol, sphingomyelin and ganglioside in a 1:1:1:0.14 molar ratio. Therefore, in some embodiments, the ganglioside-containing lecithin-based liposome comprises between 10 mol % and 50 mol % lecithin. In some embodiments, the ganglioside-containing lecithin-based liposome comprises between 1 mol % and 10 mol % ganglioside. In some embodiments, the ganglioside-containing lecithin-based liposome comprises between 10 and 50 mol % lecithin, and between 1 mol % and 10 mol % ganglioside.

In some embodiments, the ganglioside-containing lecithin-based liposome comprises:
a) between 10 mol % and 50 mol % lecithin (preferably between 20 mol % and 30 mol %),
b) between 10 mol % and 50 mol % cholesterol (preferably between 20 mol % and 30 mol %),
c) between 10 mol and 50 mol % sphingomyelin (preferably between 20 mol % and 30 mol %),
d) between 1 mol % and 10 mol % ganglioside, and
e) an anti-depressant.

Preferably, the anti-depressant is characterized by:
a) a molecular weight of between about 100 and 400 daltons, preferably between 200 and 400 daltons, more preferably between 200 and 300 daltons,
b) hydrophilicity (preferably a log P<0, more preferably a log P<−1), and
c) (optionally) a cyclic component.

In some embodiments, the ganglioside is selected from the group consisting of GM1 and GM type III. These were the gangliosides used by Taira to obtain good GI robustness and optimal G6P flux in plasma. GM1 is found in mother's milk in a concentration of between 0.02% and 0.77% of the total lipid-bound sialic acid. GM1 produces antidepressant effects in mice through a BDNF signaling cascade. Jiang, Int. J. Neuropsychopharmacology, 2016, 19(9) 1-13. It is believed that GM type III is a mixture of 20% sialic acid, and equimolar amounts of GM1 and GD1a gangliosides.

Thyroid-Releasing Hormone (TRH)

In one embodiment, the active agent is TRH. TRH is available as a supplement (Abaris). It is a clinically demonstrated as a lactation enhancer (U.S. Pat. No. 4,125,605—United States as assignee), and so should be safe for the breastfeeding infant. A rapid antidepressant response after nocturnal TRH administration has been demonstrated in patients with bipolar type I and bipolar type II major depression. Szuba, J Clin Psychopharmacol. 2005 Augist; 25(4):325-30. Therefore, TRH should be beneficial for the perinatally depressed mother. TRH has a molecular weight of about 362 daltons, is very hydrophilic (log P=−2.46), and it has a short half-life (7.6 minutes). Duntas, Thyroidology. 1991 May; 3(2):51-7. See also Bassiri, J. Clin. Invest., 52, July 1973, 1616-19. Therefore, its simple once-a-day oral delivery does not dispose itself to a relatively constant plasma concentration over the course of a day.

Therefore, TRH should pass through a ganglioside-containing lecithin-based liposome into plasma with a flux comparable to G6P, thereby allowing for once a day dosing and a constant plasma concentration.

Cyclo His-Pro

In another embodiment, the active agent suitable for once-a-day dosing through a ganglioside-containing lecithin-based liposome is Cyclo His-Pro. Cyclo His-Pro is a major TRH metabolite. It has been called “an important new tool in counteracting neuroinflammation-based degenerative pathologies” Grotelli, Intl. J. Molec. Sci., 2016, 17, 1332. Accordingly, it should be beneficial to a perinatally depressed mother whose condition is characterized by an inflammation related phenotype. It may also be useful in treating gestational diabetes, as it (with histidine) decreases blood glucose concentrations in type 2 diabetic mice (Hwang, Diabetes Obes. Metab., 2003 September, 5(5), 317-24), and protects pancreas cells from apoptosis. (Koo, J. Microbiol. Biotechnol., 2011 February, 21(2) 218-27). Cyclo His-Pro has a molecular weight of about 234 daltons, is very hydrophilic (Log P=−1.48) and has a short biphasic half-life of 1 and 33 minutes. (Koch, Biochem. Biophys. Res. Comm., 104(2), 1982, 823-9.) Therefore, it simple once-a-day oral delivery does not dispose itself to a relatively constant plasma concentration over the course of a day.

Therefore, Cyclo His-Pro should pass through a ganglioside-containing lecithin-based liposome into plasma with a flux comparable to G6P, thereby allowing for once a day dosing and a constant plasma concentration.

Taurine

Taurine is the major ingredient in popular energy drinks, and is added to many infant formulas (wikipedia). Taurine is an essential amino acid for pre-term neonates. Lourenco, Nutr. Hosp., 2002, XVII, 6,262-270. It is very hydrophilic (Log P=−3.36) and its oral administration in healthy volunteers is characterized by a short half-life of 1 hour (Ghandforoush-Sattari, J. Amino Acids, Vol. 2010, 346237). Therefore, it simple once-a-day oral delivery does not dispose itself to a relatively constant plasma concentration over the course of a day. Lastly, the antidepressant effect of chronic taurine administration has been demonstrated in rats. Toyoda, Adv. Exp. Med. Biol., 2013, 775, 29-43. Accordingly, it should be beneficial to a perinatally depressed mother.

Therefore, Taurine should pass through a ganglioside-containing lecithin-based liposome into plasma with a flux comparable to G6P, thereby allowing for once a day dosing and a constant plasma concentration.

Pyroglutamyl Leucine (PGL)

PGL is a wheat-hydrolysate, and so should be safe for an infant. PGL provides an antidepressant effect in mice through enhancing hippocampal neurogenesis (Yamamoto, Neuropeptides, 2015 June, 51, 25-9), and so may be used for a perinatally-depressed mother. PGL is also anti-inflammatory (Hirai, Life Sci., Nov. 4, 2014, 117(1) 1-6), and so should be of particular use for a perinatally-depressed mother having an inflammatory phenotype. The high number of nitrogen and COOH groups in the small molecule makes it reasonable to conclude that PGL is very hydrophilic. The fact that it is a peptide subject to rapid amidase activity makes it reasonable to conclude that PGL has a short half-life. Therefore, it simple once-a-day oral delivery does not dispose itself to a relatively constant plasma concentration over the course of a day.

Therefore, PGL should pass through a ganglioside-containing lecithin-based liposome into plasma with a flux comparable to G6P, thereby allowing for once a day dosing and a constant plasma concentration.

Carnosine

Carnosine is available as a supplement, and is highly concentrated in brain. Carnosine is very hydrophilic, with a log p=−1.19. It has a short half-life in human plasma <5 minutes (Gardner, J. Physiology, 1991, 439, 411-422). Therefore, it simple once-a-day oral delivery does not dispose itself to a relatively constant plasma concentration over the course of a day. Carnosine is credited for the antidepressant effect of chicken breast extract in rats. Tomonaga, Pharmacol Biochem Behay., June 2008, 89, 4,627-32. Accordingly, it should be beneficial to a perinatally depressed mother. Carnosine also significantly improved symptoms of autism in children. Chez, J. Child Neurol., 2002 November, 17(11), 833-7.

Because of its relatively low molecular weight and high hydrophilicity, carnosine should pass through a ganglioside-containing lecithin-based liposome into plasma with a flux comparable to G6P, thereby allowing for once a day dosing and a constant plasma concentration.

Alanyl-Glutamine (AG)

A-G is available as a supplement (Sustamine™). A-G protects against ischemia-reperfusion injury by upregulating bcl-2 (Jia, World J. Gastroenterol., Mar. 7, 2006, 12(9), 1373-8). Its ability to increase bcl-2 makes it reasonable to conclude that AG will help increase neuronal synaptic plasticity, and so makes AG an attractive candidate for antidepression therapy. AG has a plasma half-life in ICU patients was 0.26 hours (Berg, Amino Acids. 2005 November; 29(3):221-8.), and so does not remain in blood very long. Therefore, it simple once-a-day oral delivery does not dispose itself to a relatively constant plasma concentration over the course of a day. This leads one to conclude that it requires multiple dosings per day. AG has a log P=−2.15, and so is very lipophilic. AG has been used in double-blind trials in infants, (Struijs, Clin Nutr. 2013 June; 32(3):331-7) and so is likely safe for infants. The three nitrogen atoms in the molecules leads one to reasonably conclude that it is very hydrophilic.

Because of its relatively low molecular weight and high hydrophilicity, A-G should pass through a ganglioside-containing lecithin-based liposome into plasma with a flux comparable to G6P, thereby allowing for once a day dosing and a constant plasma concentration.

Glutaurine (Gamma-Glutamyltaurine) (GGT)

GGT is very hydrophilic, with a Log P=−3.36. GGT is a potent anti-epileptic in amygdala-kindled rats (Uemura, Brain Res., 1992 October, 594(2), 347-50), and it modulates excitatory neurotransmission in vitro. Varga, Neurochem. Res., 1994 March, 19(3), 243-8. Therefore, GGT is an attractive candidate for a mother who suffers from epilepsy. GGT is available as a supplement in Hungary (Litoralon). GGT is thought to affect emotional arousal and is considered to be an anti-conflict molecule. Bittner, Amino Acids, 2005 June, 28(4), 343-56.

Because of its relatively low molecular weight and high hydrophilicity, A-G should pass through a ganglioside-containing lecithin-based liposome into plasma with a flux comparable to G6P, thereby allowing for once a day dosing and a constant plasma concentration.

Non-PND Molecules

Cytarabine

Cytarabine is mainly used in the treatment of leukemia and lymphomas, where it is the backbone of induction chemotherapy. It is on the World health organization's List of Essential Medicines. The half-life of subcutaneously delivered cytarabine is 18 minutes, while its half-life in plasma via a continuous infusion is 2.1 hours. Liliemark, Semin. Oncol., 1987 June, 14 (2 supp 1) 167-71. Harris reports that its intravenously delivered half-life is 7-107 minutes. Harris. Br. J. Pharmacol., 1979 September, 8(3) 219-27. Therefore, its simple once-a-day oral delivery does not dispose itself to a relatively constant plasma concentration over the course of a day. Cytarabine is very hydrophilic, with a log P=−2.8.

Because of its relatively low molecular weight, cyclic structure and high hydrophilicity, cytarabine should pass through a ganglioside-containing lecithin-based liposome into plasma with a flux comparable to G6P, thereby allowing for once a day dosing and a relatively constant plasma concentration.

Cytarabine is a cytidine. According to Wikipedia, a cytidine is a nucleoside molecule that is formed when cytosine is attached to a ribose ring via a β-N₁-glycosidic bond. Cytidine is a component of RNA. If cytosine is attached to a deoxyribose ring, it is known as a deoxycytidine. Therefore, in some embodiments, there is provided a ganglioside-containing lecithin-based liposome comprising:
a) between 10 mol % and 50 mol % lecithin (preferably between 20 mol % and 30 mol %),
b) between 10 mol % and 50 mol % cholesterol (preferably between 20 mol % and 30 mol %),
c) between 10 mol and 50 mol % sphingomyelin (preferably between 20 mol % and 30 mol %), and
d) between 1 mol % and 10 mol % ganglioside, and
e) a cytarabine.

1-29. (canceled)

30. A method of treating a mother having postpartum depression, comprising administering to the mother a composition comprising a plurality of mixed self-assemblies comprising:
i) at least 50 wt % of a soyasaponin, and
ii) allopregnanolone.
